(19) United States
(12) Patent Application Publication
(10) Pub. No.: US 2024/0099877 A1
(43) Pub. Date: Mar. 28, 2024

(54) TONGUE STABILIZATION DEVICE WITH FRONTAL STABILIZATION

(71) Applicant: KONINKLIJKE PHILIPS N.V., EINDHOVEN (NL)

(72) Inventor: ZACHARY FORTUNE, PITTSBURGH, PA (US)

(21) Appl. No.: 18/371,202

(22) Filed: Sep. 21, 2023

Related U.S. Application Data
(60) Provisional application No. 63/408,898, filed on Sep. 22, 2022.

(51) Int. Cl. A61F 5/56

(52) U.S. Cl. CPC A61F 5/566

(57) ABSTRACT

An apparatus for stabilizing a tongue of a user during sleep. The apparatus includes a mouthpiece member structured and configured to rest on the user's teeth, and a stabilization mechanism fixed to the mouthpiece member. The stabilization mechanism includes a first stabilization assembly having a first roller member structured to rotate about a first axle and a second stabilization assembly having a second roller member structured to rotate about a second axle, the first roller member being spaced from the second roller member to form a gap between the first roller member and the second roller member. The gap is configured to receive a frontal portion of the tongue to enable the tongue to be engaged and be held by the first roller remember and the second roller member when the tongue is received in the gap.

### CROSS-REFERENCE TO RELATED APPLICATIONS

This patent application claims the priority benefit under 35 U.S.C. § 119(e) of U.S. Provisional Application No. 63/408,898, filed on Sep. 22, 2022, the contents of which are herein incorporated by reference.

### 1. FIELD OF THE INVENTION

The disclosed concept relates generally to the treatment of obstructive sleep apnea (OSA) and/or snoring, and, in particular, to a device for stabilizing the tongue of an individual while sleeping in order to treat and/or prevent OSA and/or snoring.

### 2. BACKGROUND OF THE INVENTION

Obstructive sleep apnea (OSA) is a condition in which an individual's airway is either partially or fully blocked during sleep. Blockage typically occurs when the individual's tongue falls back towards the airway, blocking airflow. This obstruction can lead to a complete halt in airflow, causing the individual to stop breathing briefly as they sleep. These apnea events often cause arousals from sleep and in turn leave those who suffer from OSA with a night of heavily disrupted, low quality sleep. The apnea/hypopnea index (AHI) is the number of these arousals per hour during sleep. This metric includes apnea events, which are periods of no breathing, and hypopnea events, which are periods or abnormally show and/or shallow breathing. Individuals with severe OSA have an AHI of over 30.

OSA may also be caused by the soft palate tissue in the throat relaxing and decreasing the diameter of the airway. As air passes through this constricted airway, the tissue can begin to vibrate as the individual breaths, causing snoring. This can make for a poor sleeping experience for the patient's bed partner. Many times, OSA patients experience a combination of both soft tissue constriction and tongue blockage.

Snoring is a hallmark symptom and sometimes an indicator of OSA. Snoring is caused when the airway relaxes and closes partially or is slightly blocked by the relaxed muscles and/or the tongue. Air passing through the constricted airway causes the soft tissue to vibrate. Snoring is the sound produced by this vibration. Snoring most often occurs when one is laying on their back during sleep. This position makes it possible for the tongue to fall back into the airway due to gravity. Rolling over or changing sleeping position can help lessen or even eliminate snoring but most snorers would rather not be disturbed during their sleep to do this. A more comfortable and permanent solution would be to stabilize the tongue. This way, the patient of this proposed device can sleep in whatever position is comfortable without the tongue falling into and blocking the airway.

### SUMMARY OF THE INVENTION

These needs, and others, are met by an apparatus for stabilizing the tongue of a user during sleep. The apparatus includes a mouthpiece member structured and configured to rest on the user's teeth (e.g., lower bridge), and a stabilization mechanism fixed to the mouthpiece member. The stabilization mechanism includes a first stabilization assembly having a first roller member structured to rotate about a first axle and a second stabilization assembly having a second roller member structured to rotate about a second axle, the first roller member being spaced from the second roller member to form a gap between the first roller member and the second roller member. The gap is configured to receive a frontal portion of the tongue to enable the tongue to be engaged and be held by the first roller remember and the second roller member when the tongue is received in the gap.

These needs, and others, are met by a method of stabilizing the tongue of a user during sleep that includes resting an apparatus on the user's teeth (e.g., lower bridge) that includes a mouthpiece member and stabilization mechanism fixed to the mouthpiece member. The stabilization mechanism includes a first stabilization assembly having a first roller member structured to rotate about a first axle and a second stabilization assembly having a second roller member structured to rotate about a second axle, the first roller member being spaced from the second roller member to form a gap between the first roller member and the second roller member. The method also includes inserting a frontal portion of the tongue into the gap and engaging and holding the tongue by the first roller remember and the second roller member.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following description of the preferred embodiments when read in conjunction with the accompanying drawings in which:

FIG. 1 is an isometric view of a tongue stabilization device according to an exemplary embodiment of the disclosed concept;

FIG. 2 is an isometric view of a mouthpiece member forming part of the tongue stabilization device of FIG. 1;

FIG. 3 is an isometric view of a tongue stabilization mechanism forming part of the tongue stabilization device of FIG. 1;

FIG. 4 is an and you of a roller member that forms part of the tongue stabilization device of FIG. 1; and

FIGS. 5A and 5B illustrate exemplary operation of the tongue stabilization device of FIG. 1.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As used herein, the statement that two or more parts or components “engage” one another shall mean that the parts exert a force against one another either directly or through one or more intermediate parts or components.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

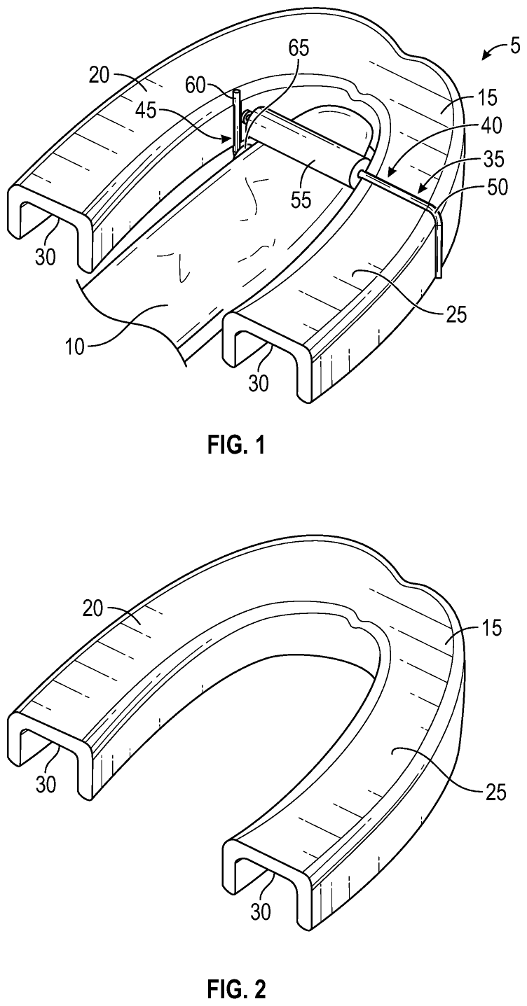

FIG. 1 is a schematic diagram of a tongue stabilization device 5 according to an exemplary embodiment of the disclosed concept. Tongue stabilization device 5 is structured and configured to hold the frontal portion of an individual's tongue 10 (and thus stabilize tongue 10) while the individual is sleeping so as to treat and/or prevent OSA and/or snoring. Holding the frontal portion of tongue 10 as just described helps to prevent a gag reflex from the individual during use. In addition, when held in this manner, tongue 10 will remain inside the individual's mouth. This is in contrast to certain prior art anti-snore devices wherein the tongue protrudes from the mouth and thus leads to discomfort of and/or drooling by the user.

As seen in FIG. 1, tongue stabilization device 5 includes a mouthpiece number 15 that is structured and configured to be worn by the individual on the individual's lower teeth (i.e., lower bridge). Mouthpiece number 15 is shown separately in FIG. 2 and includes a left portion 20 and a right portion 25. Each portion 20, 25 includes a groove or trench 30 that is structured and configured to receive therein the lower teeth of the individual in order to hold mouthpiece number 15, and thus tongue stabilization device 5, in place during use. In the exemplary embodiment, mouthpiece number 15 is made of a material that may be selectively formed, such as by heating/molding, to conform to the teeth of the individual in a manner similar to a mouthpiece that is used in sports activities.

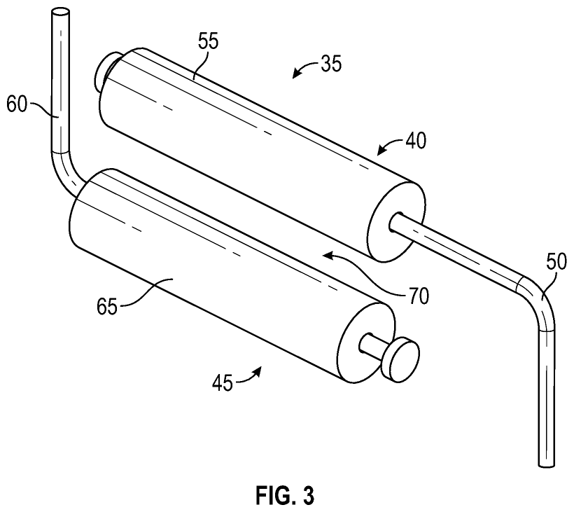

Tongue stabilization device 5 also includes a tongue stabilization mechanism 35 that, as described in more detail herein, is structured and configured to hold the front portion of tongue 10 and thereby stabilize tongue 10 in order to treat and/or prevent OSA and/or snoring while the individual is sleeping. Tongue stabilization mechanism 35 includes a top stabilization assembly 40 and a bottom stabilization assembly 45, each of which is shown separately in FIG. 3. As seen in FIG. 3, top stabilization assembly 40 includes an axle member 50 made of a rigid material such as metal and a roller member 55 made of a more flexible/compressible material such as silicone or memory foam. Roller member 55 is rotatably received and held on a distal end of axle 50. Similarly, bottom stabilization assembly 45 includes an axle member 60 made of a rigid material such as metal and a roller member 65 made of a more flexible/compressible material such as silicone or memory foam. Roller member 65 is rotatably received and held on a distal end of axle 60. In addition, in the illustrated non-limiting exemplary embodiment, axle members 50 and 60 each include an enlarged portion at the terminal distal end thereof to prevent the associated roller member 55, 65 from sliding off of the axle member 50, 60. In this manner, roller assembly 55 is structured and configured to rotate about the distal end of axle 50 and roller member 65 is structured and configured to rotate about to the distal end of axle 60.

Referring again to FIG. 1, tongue stabilization mechanism 35 is fixed to mouthpiece member 50 in a manner wherein a gap 70 (FIG. 3) is provided between roller members 55 and 65. The gap 70 is sized so as to receive tongue 10 therethrough. In addition, gap 70 is sized such that, in the exemplary embodiment, tongue 10 will take on the shape of gap 70 when tongue 10 is inserted through gap 70. More specifically, as seen in FIG. 1, in the illustrated non-limiting exemplary embodiment, top stabilization assembly 40 is fixed to the right side of mouthpiece member 15 by affixing the proximal portion of axle 50 to the right side 25 of mouthpiece member 15. Similarly, bottom stabilization assembly 45 is fixed to the left side 20 of mouthpiece member 15 by affixing the proximal portion of axle 60 to the left side 20 of mouthpiece number 15. Any suitable affixation method may be employed to fix tongue stabilization mechanism 35 in place, such as an appropriate adhesive or such as by over-molding the tongue stabilization mechanism 35 with mouthpiece member 15. In this way, axles 50, 60 have substantially zero degrees of motion. In an alternative embodiment of the disclosed concept, axles 50 and 60 may be longer than shown and be affixed to both the left side 20 and the right side 25 of mouthpiece member 15 in order to fix tongue stabilization mechanism 35 in place on mouthpiece member 15.

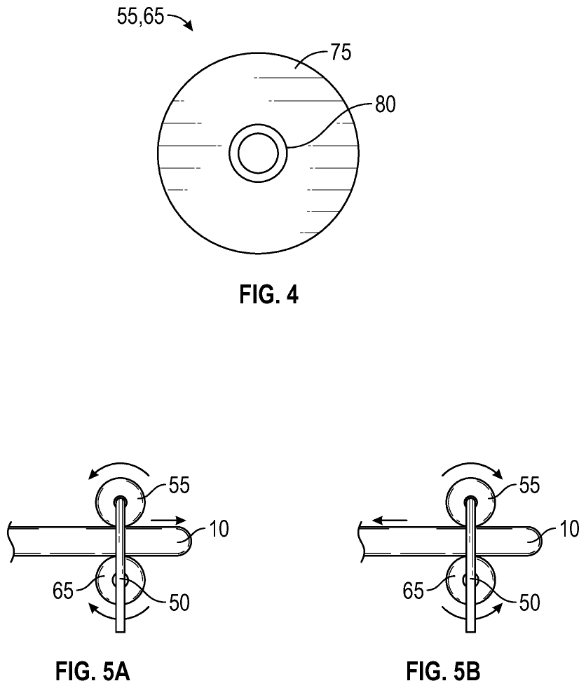

FIG. 4 is an end view of the roller member 55, 65 according to a non-limiting exemplary embodiment of the disclosed concept. As seen in FIG. 4, each roller member 55, 65 in this embodiment comprises a hollow cylindrical portion 75 that is made of the flexible material described above, wherein a coating or casing 80 is provided on (i.e., lines) the inner surface of hollow cylindrical portion 75. Coating or casing 80 in this exemplary embodiment is made of a material, such as metal (e.g., the same material as axles 55 and 60), that facilitates the rotation of each roller member 55, 65 about its associated axle 50, 60. More specifically, the coating or casing 80 ensures that the axis of rotation remains rigid at a 180° angle and further ensures that the material of roller members 55 and 60 will not interfere with the axis of rotation when stressed (e.g., when tongue 10 is inserted as described herein).

In operation, before going to bed, the individual inserts tongue stabilization device 5 into his or her mouth on the lower bridge. Tongue 10 is then inserted into gap 70. When so inserted, roller member 55 will rotate counterclockwise and roller member 65 will rotate clockwise as shown in FIG. 5A to allow tongue 10 to move forward through gap 70 and be positioned and held in the manner shown in FIG. 1. The individual is able to sleep comfortably with tongue stabilization device 5 in this configuration, with tongue 10 being stabilized so as to prevent OSA and/or snoring. At the end of the sleeping period, tongue 10 is then removed from gap 70. During such removal, roller member 55 will rotate clockwise and roller member 65 rotate counterclockwise as shown in FIG. 5B.

In the exemplary embodiment, the material of roller members 55 and 65 will have a coefficient of friction that is high enough to prevent tongue 10 from passively slipping out but low enough to allow tongue 10 to be easily removed by the individual when he or she wishes to disengage with tongue stabilization device 5. In the non-limiting exemplary embodiment, such a coefficient of friction may be accomplished by providing an appropriate superficial coating to the outside of cylindrical portion 75 of each roller member 55, 60. Roller members 55 and 65 will themselves be flexible enough to allow for gentle torsion inflection of tongue 10 in response to nocturnal breeding maneuvers.

In a further exemplary embodiment, axles 50 and 60 and corresponding inner portions of roller members 55, 65 will feature a series of micro-grooves, teeth or bumps that will engage one another and introduce friction when roller members 55, 65 are caused to rotate in the direction shown in FIG. 5B when removing tongue 10. For this embodiment, the micro-grooves, teeth or bumps may be provide as part of coating or casing 80 and as part of the outer surface of axles 50, 60. This feature will prevent tongue 10 from slipping out of gap 70 when the individual is asleep. The level of friction (as measured by the coefficient of friction) to be introduced by either roller members 55 and 65 themselves and/or by way of introduction of the microgrooves, teeth or bumps as just described will, in the exemplary embodiment, be in the range of about 0.3 to 0.5. This determination is based on calculations using the average weight of a tongue and an estimate of the amount of force that is required to passively move the tongue back.

The disclosed concept thus provides a novel tongue stabilization device that is configured to hold and secure the frontal portion of the individual's tongue while the individual is sleeping. As a result, holding the tongue forward in this manner will help to prevent OSA and/or snoring by helping to facilitate an open airway for the individual while sleeping.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof

What is claimed is:

1. An apparatus for stabilizing a tongue of a user during sleep, comprising:
a mouthpiece member structured and configured to rest on the user's teeth; and
a stabilization mechanism fixed to the mouthpiece member, the stabilization mechanism including a first stabilization assembly having a first roller member structured to rotate about a first axle and a second stabilization assembly having a second roller member structured to rotate about a second axle, the first roller member being spaced from the second roller member to form a gap between the first roller member and the second roller member, wherein the gap is configured to receive a frontal portion of the tongue to enable the tongue to be engaged and be held by the first roller remember and the second roller member when the tongue is received in the gap.

2. The apparatus according to claim 1, wherein the mouthpiece member structured and configured to rest on the user's lower bridge.

3. The apparatus according to claim 1, wherein the first axle and the second axle are fixed to the mouthpiece member.

4. The apparatus according to claim 3, wherein the first axle is fixed to a right side of the mouthpiece member and the second axle is fixed to a left side of the mouthpiece member.

5. The apparatus according to claim 1, wherein the first axle and the second axle are made of a rigid material, and wherein the first roller member and the second roller member each include a flexible/compressible material.

6. The apparatus according to claim 5, wherein the rigid material comprises a metal.

7. The apparatus according to claim 5, wherein the flexible/compressible material comprises silicone or memory foam.

8. The apparatus according to claim 1, wherein the first roller member and the second roller member are structured to operate together so as roll with a coefficient of friction of 0.3 to 0.5.

9. The apparatus according to claim 1, wherein the first roller member and the second roller member each comprise a hollow cylindrical portion that is made of a flexible material and a coating or casing made of a rigid material that lines an inner surface of hollow cylindrical portion.

10. The apparatus according to claim 9, wherein an outer surface of the first axle and the second axle and each coating or casing each include a plurality of microgrooves, teeth or bumps.

11. A method of stabilizing a tongue of a user during sleep, comprising:
resting on the user's teeth an apparatus that includes a mouthpiece member and a stabilization mechanism fixed to the mouthpiece member, the stabilization mechanism including a first stabilization assembly having a first roller member structured to rotate about a first axle and a second stabilization assembly having a second roller member structured to rotate about a second axle, the first roller member being spaced from the second roller member to form a gap between the first roller member and the second roller member; and
inserting a frontal portion of the tongue into the gap and such that the tongue is engaged and held by the first roller remember and the second roller member.

12. The method according to claim 11, wherein resting comprises resting the mouthpiece member on the user's lower bridge.

13. The method according to claim 11, wherein the first axle and the second axle are fixed to the mouthpiece member.

14. The method according to claim 13, wherein the first axle is fixed to a right side of the mouthpiece member and the second axle is fixed to a left side of the mouthpiece member.

15. The method according to claim 11, wherein the first axle and the second axle are made of a rigid material, and wherein the first roller member and the second roller member each include a flexible/compressible material.

16. The method according to claim 15, wherein the rigid material comprises a metal.

17. The method according to claim 15, wherein the flexible/compressible material comprises silicone or memory foam.

18. The method according to claim 11, wherein the first roller member and the second roller member roll with a coefficient of friction of 0.3 to 0.5 when the tongue is inserted onto the gap.

19. The method according to claim 11, wherein the first roller member and the second roller member each comprise a hollow cylindrical portion that is made of a flexible material and a coating or casing made of a rigid material that lines an inner surface of hollow cylindrical portion.

20. The method according to claim 19, wherein an outer surface of the first axle and the second axle and each coating or casing each include a plurality of microgrooves, teeth or bumps.
